SEQUENCE LISTING (2 sequences)

SEQ ID NO: 1 - protein, 11 aa, Unknown
  Substance P
RPKPQQFFGLM

SEQ ID NO: 2 - protein, 36 aa, Unknown
  Neuropeptide Y
YPSKPDNPGEDAPAEDMARYYSALRHYINLITRQRY

(19) United States
(12) Patent Application Publication
(10) Pub. No.: US 2015/0226737 A1
(43) Pub. Date: Aug. 13, 2015

(54) COMPLEX COMPRSING BEAD PARTICLE INCLUDING QUANTUM DOT LAYER AND METHOD OF DIAGNOSING MYOCARDIAL INFARCTION-RELATED DISEASE BY USING THE COMPLEX

(71) Applicant: KOREA INSTITUTE OF SCIENCE AND TECHNOLOGY, Seoul (KR)

(72) Inventors: Min-Jung KANG, Seoul (KR); Kyoungja WOO, Seoul (KR); Hyojeong HAN, Seoul (KR); Byung Hwa JUNG, Seoul (KR)

(21) Appl. No.: 14/580,724

(22) Filed: Dec. 23, 2014

(30) Foreign Application Priority Data
Feb. 7, 2014 (KR) 10-2014-0014453

(51) Int. Cl. G01N 33/552; G01N 33/58; G01N 33/68

(52) U.S. Cl. CPC G01N 33/552; G01N 33/6893; G01N 33/588; G01N 2800/324

(57) ABSTRACT

Provided are a complex including a quantum dot layer-containing bead particle and an agent for detecting or analyzing a target material; a composition including the complex for detecting the target material or for diagnosing myocardial infarction-related disease; and a method of diagnosing myocardial infarction-related disease by using the complex.

### RELATED APPLICATION

This application claims the benefit of Korean Patent Application No. 10-2014-0014453, filed on Feb. 7, 2014, in the Korean Intellectual Property Office, the disclosure of which is incorporated herein in its entirety by reference.

### BACKGROUND

1. Field

One or more embodiments of the present invention relate to a complex including a quantum dot layer-containing bead particle and an agent for detecting or analyzing a target material, a composition for analyzing a target protein including the complex, a composition for diagnosing myocardial infarction-related disease by using the complex, and a method of diagnosing myocardial infarction-related disease by using the complex.

2. Description of the Related Art

Diagnosis of acute myocardial infarction is currently carried out by diagnosing chest pain, performing electrocardiography, or observing changes in concentrations of hormones (e.g., CK-KB, myoglobin, cardiac troponin, etc.) via blood tests, and definite diagnosis may be confirmed through cardiac angiography. In most cases, a patient visits a hospital after occurrence of myocardial infarction, and thereby the patient has to pay high expense for diagnosis or treatment of myocardial infarction. Although cardiac troponin is considered as a gold standard in the diagnosis of acute myocardial infarction, there are difficulties in the early diagnosis because cardiac troponin has specificity of less than 80% and is released into the blood 6 hours after occurrence of a heart attack. In this regard, when the cardiac troponin is used for diagnosis, the real acute myocardial infarction by cardiac troponin may be possibly diagnosed as a negative by cardiac troponin. Thus, physical diagnostic tests including electrocardiography, magnetic resonance imaging (MRI), and X-ray are still mainly carried out for diagnosing acute myocardial infarction. In addition, upon the occurrence of acute myocardial infarction, its effects are so fatal that the importance of the early diagnosis is more emphasized. Accordingly, the discovery of diagnostic peptide markers has been accelerated, and an example of well-known markers related to cardiovascular disease is N-terminal proBNP (NT-proBNP). It is known that concentration of NT-proBNP increases in a patient with acute myocardial infarction, as time passes. However, in consideration of concentration of NT-proBNP in blood, NT-proBNP is more commonly known as a marker for congestive heart failure (CHF). In addition, as in the case of troponin, NT-ProBNP is released into the blood upon apoptosis of heart cells, and thus, it is difficult to use NT-ProBNP in the early diagnosis of acute myocardial infarction. Methods using chest pain diagnosis, electrocardiography, and cardiac angiography also are not considered as suitable methods for the early diagnosis. Thus, it is now necessary to discover a blood biomarker for the early diagnosis, a method with high specificity and sensitivity for diagnosing acute myocardial infarction, and a method for diagnosing myocardial infarction-related disease.

### SUMMARY

One or more embodiments of the present invention include a complex including a quantum dot layer-containing bead particle and an agent for detecting or analyzing a target material.

One or more embodiments of the present invention include an analysis composition for a target material, an immunoassay kit for a target material, a diagnostic composition for myocardial infarction-related disease, in which the analysis composition, the immunoassay kit and the diagnostic composition include the complex including the quantum dot layer-containing bead particle and the agent for detecting or analyzing the target material.

One or more embodiments of the present invention include a method of manufacturing the complex including the complex including the quantum dot layer-containing bead particle and the agent for detecting or analyzing the target material.

One or more embodiments of the present invention include a method of diagnosing myocardial infarction-related disease by using the complex including the quantum dot layer-containing bead particle and the agent for detecting or analyzing the bead particle and the target material.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1A illustrates Schematic diagram of Parylene-A and Substance P (SubP) (P) coating process of 96-well polystyrene plates;

FIG. 1B illustrates labeling process of the anti-SubP antibody with SQS using glutaraldehyde as a cross linker;

FIG. 1C illustrates experimental flow for competitive SQSLISA;

FIG. 2A shows the results of PL spectra of QD-MPA(Quantum Dot-mercaptopropionic acid), QD-ODA(Quantum Dot-Octadecyl amine), and SQS at a constant QD concentration;

FIG. 2B shows TEM image of SQS prepared with intermittent sonication;

FIG. 3 is a graph showing comparison of parylene A-coated and non-coated plates for SubP immunoassays, in which triplicate samples were analyzed;

FIG. 4A shows the results of optimization of (a) the ratio of SQS to antibody;

FIG. 4B shows the results of detection sensitivity according to various types and concentrations of blocking reagent;

FIG. 4C shows the results of incubation time of SubP for binding on plate;

FIG. 5A is a graph showing Standard curve and linearity of direct SQSLISA in the range of 1-10000 pg/mL of SubP;

FIG. 5B is a graph showing Standard curve and linearity of competitive SQSLISA in the range of 1-10000 pg/mL of SubP;

FIG. 5C is a graph showing Standard curve and linearity of commercial ELISA in the range of 39-2500 pg/mL;

FIG. 6 shows the results of concentration of Neuropeptide Y (NpY) in sera of AMI (acute myocardial infarction), UA (unstable angina), SA (stable angina), and healthy controls measured by commercial ELISA;

FIG. 7 shows the results of concentration of SubP in sera of AMI (acute myocardial infarction), UA (unstable angina), SA (stable angina), and healthy controls measured by commercial ELISA;

FIG. 8 shows the results of concentration of SubP for clinical samples with AMI and healthy controls measured by (a) the commercial ELISA kit, (b) the competitive SQSLISA, ** indicates P<0.001 compared to healthy controls;

FIG. 9 shows the Passing and Bablock to show linearity and deviation from linearity between the commercial ELISA and competitive SQSLISA; and

FIG. 10 is ROC curves of clinical samples for AMI patients against healthy controls measured by (a) the commercial ELISA kit and (b) competitive SQSLISA.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be constructed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

One aspect of the present invention provides a complex including a quantum dot layer-containing bead particle and an agent for detecting or analyzing a target material.

The bead particle is a spherical-shaped particle containing a quantum dot layer, and for example, the quantum dot layer may be located inside the bead particle. The term “quantum dot” as used herein may refer to a nanometer-sized particle having a zero-dimensional structure with distinctive optical and electronic properties, and for example, the quantum dot may be a nanometer-sized semiconductor crystal. The quantum dot may consist of a core body, a shell unit surrounding the core body, and a polymer coating layer coating the shell unit. Detailed types of the quantum dot are not particularly limited in the present invention, and any material may be used without limitation if it has available biocompatibility in uses for bio-imaging techniques. The core body of the quantum dot may consist of, for example, cadmium selenide (CdSe), cadmium telluride (CdTe), cadmium sulfide (CdS), zinc selenide (ZnSe), zinc oxide (ZnO), or zinc sulfide (ZnS). These components may emit fluorescence, which is much stronger than fluorescence emitted by a typical fluorescent material, at a narrow wavelength band. The term “quantum dot layer” as used herein refers to a quantum dot sheet or a quantum dot film, which is formed by electrical force, magnetic force, chemical bonding force, or external or internal force of plurality of quantum dots. Here, the formation of a quantum dot sheet or a quantum dot film may include not only a case of forming a complete quantum dot film, but also a case of being located on a concentric sphere and present thereon without forming a complete quantum dot film.

The bead particle may comprise quantum dots that are bound to a concentric sphere located close to an interior to a surface of the bead particle by radial electrostatic attraction. For example, each quantum dot is located in the same radial distance from the center of the bead particle and forms a single-layer sphere shell, thereby doping the interior of the bead particle. In the present invention, the single-layer sphere shell formed of the quantum dots may provide a doping layer that has uniform density without occurring oxidation reaction of the quantum dots through doping of the quantum dots by electrostatic attraction, wherein use of an organic polymer is excluded. Here, the quantum dots are present in a single-layer concentric sphere shape, and in this regard, self-extinction phenomenon of the quantum dots may be minimized and fluorescence amplified by resonance with cavity of the bead particle may be emitted.

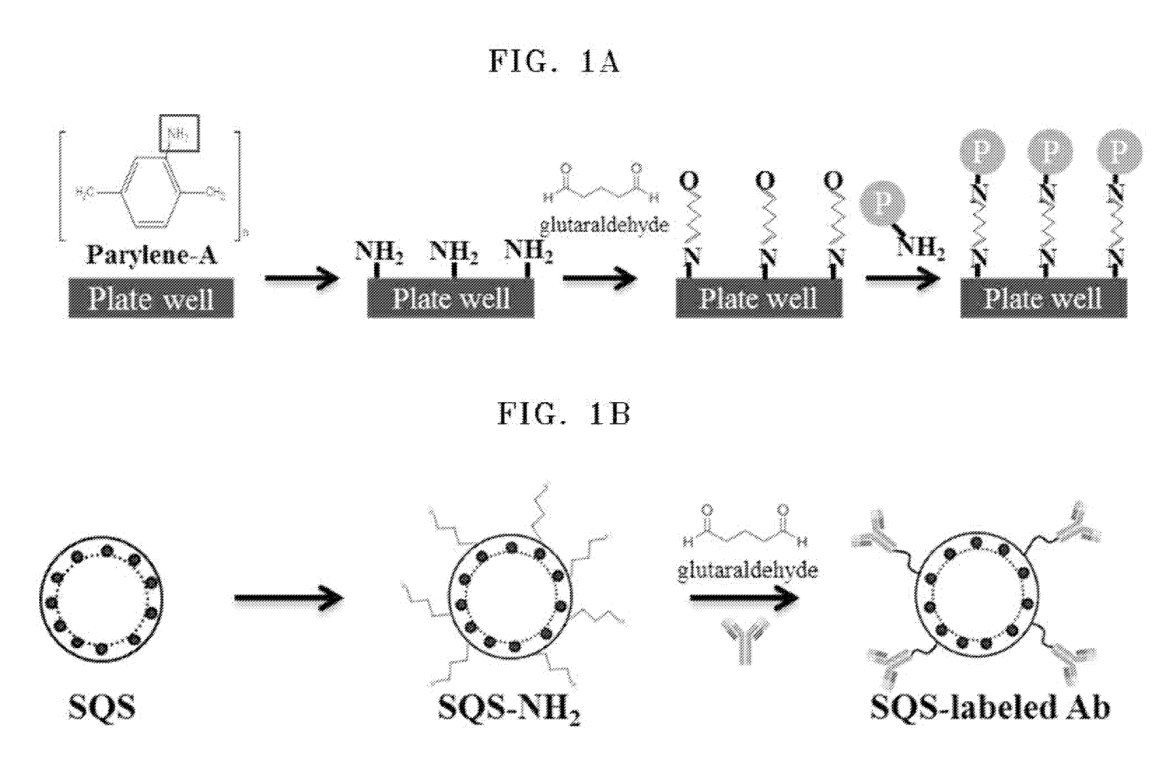

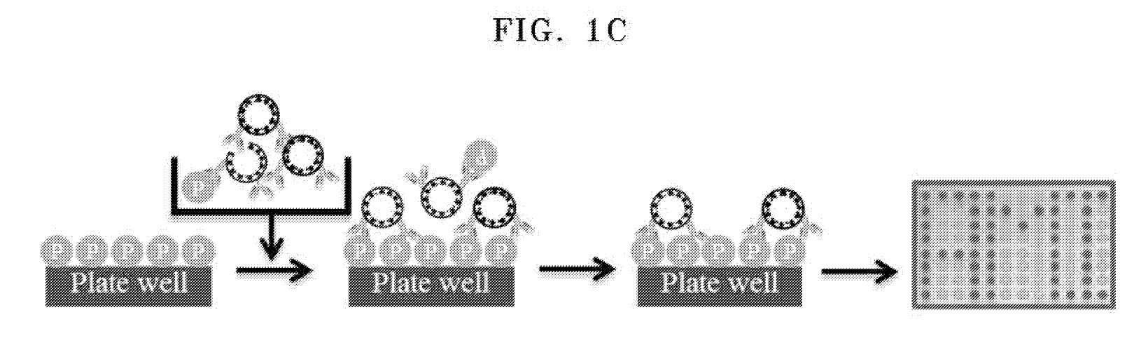

The concentric sphere may have a radius (r) that is more than 0.5 times and less than 1 time, more than 0.7 times and less than 1 time, or more than 0.9 times and less than 1 time of a distance (radius, R) from the center to the surface of the bead particle. For example, the concentric sphere may be located the interior of the bead particle adjacent to the surface of the bead particle. When the concentric sphere has the radius r less than 0.5 times of the radius R, the quantum dot layer may be formed in a too deep position within the bead particle, and accordingly, the intensity of fluorescence emitted out of the bead particle becomes too weak. The radius r less than 1 time of the radius R indicates that the quantum dots are prevented from being exposed out of the bead particle. That is, the quantum dots are located inside the bead particle, and the bead particle may form a porous layer thereby (see FIG. 1B). The porous layer may be formed of homogenous materials with those forming the interior of the bead particle. Alternatively, the porous layer may be formed of heterogeneous materials from those forming the interior of the bead particle. The quantum dots are encased by the porous layer to be enclosed within the bead particle. In this regard, such enclosed quantum dots have advantages that light stability and durability of the quantum dots are enhanced compared to quantum dots that are present alone without forming a porous layer, and at the same time, the intensity of light emission is further amplified by a resonance coupling phenomenon of the quantum dot in the cavity of the porous layer.

The bead particle may have a diameter, for example, in a range of about 80 to about 300 nm, about 80 to about 250 nm, about 80 to about 200 nm, about 80 to about 150 nm or about 80 to about 100 nm. When the bead particle has a diameter of 80 nm or less, the bead particle containing the quantum dot layer cannot avoid aggregation between them during synthesis because of the high surface energy resulting from their small size and aggregation increases the light scattering effect. When the bead particle has a diameter of 300 nm or more, photoluminescence (PL) of the bead particle may diminish, and, consequently, the bead particle may have lower sensitivity. When the bead particle has a diameter in a range of about 80 to about 100 nm, the quantum dot show appropriate light-emitting features because of the high surface energy resulting from its small size and the reduction of aggregation.

The bead may be formed of at least one material selected from the group consisting of silica, titanium, zirconia, and zeolite, and specifically, may be formed of silica. In addition, the bead particle is not particularly limited as long as the bead particle is form of an inorganic material that has a high refractive index.

Regarding the complex of the present invention, the agent for detecting or analyzing a target material may bind to the bead particle, and more particularly, may bind to the surface of the bead particle. The term “target material” as used herein may refer to a protein, a nucleic acid, a peptide, a cell, an intracellular organelle, or other physiologically active materials, as a subject for detection or analysis using the complex. The term “agent for detecting or analyzing a target material” as used herein may refer to a material capable of specifically binding to a target material or a fragment thereof. Alternatively, the agent for detecting or analyzing a target material may refer to a target material itself or a fragment thereof. The term “fragment” as used herein refers to a portion of the target material which is physically, enzymatically, or chemically cleaved. For example, in the case of a protein, a fragment of the protein may be any fragment that is cleaved by a protease or that is chemically cleaved. The expression “binding specifically to a target material or a fragment thereof” as used herein refers that the agent included in the complex selectively binds to a target material or a fragment thereof, and does not actually bind to a material other than the target material. The term “material specifically binding to a target material” as used herein may refer to a protein, a nucleic acid, a peptide, or a derivative thereof, which specifically binds to a target material. For example, the material specifically binding to a target material may refer to an antibody specific to a target material, or a peptide including a domain that is specific to a target material. The term “antibody” as a term known in the art refers to a specialized immunoglobulin which is directed toward an antigenic site (or epitope). The antibody may be in the form of a polyclonal antibody, a monoclonal antibody, and a recombinant antibody, and in this regard, all the immunoglobulin antibodies fall within the range of the antibody of the present invention. The antibody may be in a complete form composed of two full-length light chains and two full-length heavy chains. In addition, the antibody may be a special antibody including a chimeric antibody, a humanized antibody, and a human antibody. The term “partial peptide having a binding domain specific to a target material” as used herein refers to a polypeptide that does not have a complete antibody structure, but has an antigen-binding site, i.e., a binding domain, which is directed toward an antigenic site (or epitope). For example, the partial peptide includes a functional fragment of an antibody molecule rather than an antibody in a complete form composed of two light chains and two heavy chains. The functional fragment of the antibody molecule mean a fragment retaining at least antigen-binding functionality, and examples thereof include Fab, F(ab′), F(ab′)₂, and Fv. The partial peptide may include at least 7 amino acids, for example, at least 9 amino acids or at least 12 amino acids.

The agent for detecting or analyzing the target material may be bound to the surface of the bead particle via a covalent bond, a hydrogen bond, an ionic bond, or a chemical bond such as a Van der Waals bond, etc. For example, the agent may be bound to the surface of the bead particle via a covalent bond. The covalent bond may include an amide bond, a disulfide bond, phosphorylation, or an ester bond, or may refer to oxim formation. For example, the covalent bond may be amide bond.

In order to bind the bead particle to the agent, the surface of the bead particle may contain a reactive group, or the surface of the bead particle may be activated to allow a reactive group to be introduced thereto. The expression “the surface of the bead particle may be activated” as used herein refers that a reactive group, which is capable of forming a chemical bond as well as a covalent bond, is introduced to the surface of the bead particle so as to bind to other materials, and also refers to ‘surface modification’. The term “surface modification” as used herein refers to transformation or alteration of a surface to facilitate binding with other materials without causing any change in fundamental physical properties of a subject for modification. When the surface of the bead particle is activated, the surface may include a reactive group, amino group. In some embodiments, a surface of a silica bead particle may include for example, a hydroxyl group, a carboxyl group, a thiol group, a sulfonyl group, or an amino group (see FIG. 1B).

The bead particle may be directly bound to the agent via a covalent bond, or may be bound to the agent via a suitable linker-mediated covalent bond. The term “linker” as used herein refers to a material that links the bead particle with the agent that specifically binds to a target material, and an example of the linker include a compound including a reactive functional group, a sugar, a peptide, or a nucleic acid. In some embodiments, glutaraldehyde may act as a linker.

The target material may be, for example, Substance P (SubP), Neuropeptide Y (NpY), and N-terminal pro-benign natriuretic peptide (NT-proBNP). The term “Sub P” as used herein refers to a mammalian tachykinin neuropeptide. In addition, SubP may have a protein sequence of SEQ ID NO: 1. The term “NpY” as used herein refers to a peptide that is widely distributed throughout the central nervous system. In addition, NpY may have a protein sequence of SEQ ID NO: 2 (see Table 1 below). The term “NT-proBNP” as used herein refers to N-terminal of the prohormone brain natriuretic peptide which is a 76 amino acid N-terminal inactive protein that is cleaved from proBNP to release brain natriuretic peptide. In some embodiments, the complex of the present invention may include SubP, NpY, NT-proBNP or a fragment thereof. Alternatively, the complex of the present invention may include an antibody, a nucleic acid, a peptide, or a derivative thereof, which specifically binds to SubP, NpY, NT-proBNP or a fragment thereof, and in addition, these materials may be bound to the surface of the bead particle.

TABLE 1

| Pep- | | SEQ ID |
|---|---|---|
| tide | Sequence information | NO. |
| SubP | RPKPQQFFGLM | SEQ ID |
| | | NO: 1 |
| NpY | YPSKPDNPGEDAPAEDMARYYSALRHYINLITRQRY | SEQ ID |
| | | NO: 2 |

Another aspect of the present invention provides an analysis composition for a target material, including a complex that includes a quantum dot layer-containing bead particle and an agent for detecting or analyzing a target material.

A description of the complex included in the analysis composition is the same as described above. The term “analyzing a target material” as used herein refers to all actions required to perform studies on detection or quantification of a target material. The analysis composition may further include a material required to perform analysis such as detection or quantification of a target material. For example, the material required to perform such analysis may be an antibody or a fragment thereof, a reagent for cell staining, or a buffer.

Another aspect of the present invention provides an immunoassay kit for a target material, including a complex that includes a quantum dot layer-containing bead particle and an agent for detecting or analyzing a target material.

A description of the complex included in the immunoassay kit is the same as described above. The term “immunoassay kit” as used herein refers to an instrument capable of analyzing a target material according to an immunological analysis method such as detection or quantification of a target material, i.e., a method of analyzing a target material based on binding functionality of an antibody with respect to a target material. The immunoassay kit may include, for example, a target material or an antibody of the target material as the agent included in the complex for detecting or analyzing the target material. The immunoassay kit may additionally include at least one selected from other components, a composition with other components, a solution, or an apparatus, suitable for the method of analyzing the target material. In some embodiments, the immunoassay kit may include, for example, a complex including an antibody specifically binding to a target material, a substrate for immunological detection of an antibody, a suitable buffer solution, or a complex to which a secondary antibody is bound. Examples of the substrate include a nitrocellulose film, a 96-well polyvinyl plate, a 96-well polystyrene plate, and a glass slide.

The analysis composition for the target material and the immunoassay kit for the target material immunoassay use a quantum dot as a detector for the target material. In this regard, based on fluorescent images formed by quantum dots, the presence of the target material may be identified, and quantitative analysis and separation of the target material may be performed more excellently. In particular, due to high sensitivity of the quantum dots, the quantum dots may be used excellently for analysis of small amounts of the target material.

In some embodiments, the immunoassay kit may comprise a complex comprising a quantum dot layer-containing bead particle and an antibody which specifically binds to a target material, and a substrate.

A description of the bead particle included in the immunoassay kit is the same as described above.

The bead particle may have a diameter, for example, in a range of about 80 to about 300 nm, about 80 to about 250 nm, about 80 to about 200 nm, about 80 to about 150 nm or about 80 to about 100 nm.

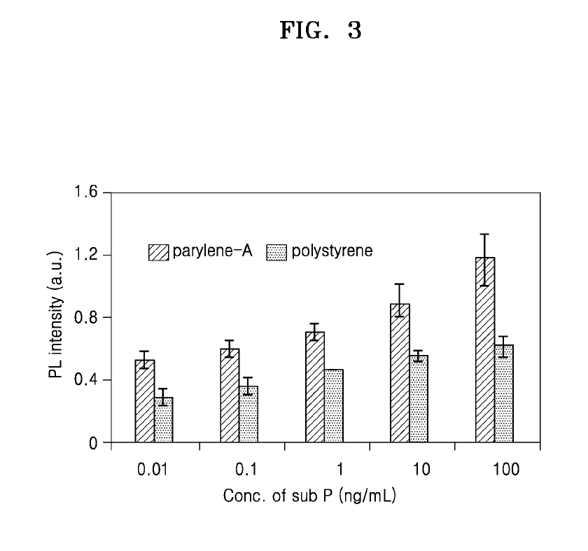

The substrate may be coated with Parylene A. In example, Parylene-A coated plates exhibited approximately 2-fold higher PL intensity than the polystyrene plates in the range of 0.01-100 ng/mL of SubP (See FIG. 3).

The complex included in the immunoassay kit may be prepared by reacting a quantum dot layer-containing bead particle and antibody at a moral ratio of about 1:150 to about 1:190, about 1:160 to about 1:190, about 1:170 to about 1:190, about 1:180 to about 1:190 or about 1:188.

The immunoassay kit further comprises a blocking agent, wherein the blocking agent is BSA in a range between about 0.5 mg/mL to about 2 mg/mL, about 0.5 mg/mL to about 1.5 mg/mL, about 0.7 mg/mL to about 1.3 mg/mL or about 0.8 mg/mL to about 1.2 mg/mL.

The target material may be Substance P (Sub P) Neuropeptide Y (NpY), N-terminal pro-benign natriuretic peptide (NT-proBNP), and, consequently, the immunoassay kit may be used for the diagnosis of acute myocardial infarction.

Compared to commercial ELISA, The immunoassay kit according to the present invention has advantages as follows. First, no enzyme is necessary. Second, reproducibility is not affected by inhibition of enzyme activity from different matrixes. Third, the immunoassay kit according to the present invention shows no photo bleaching effect, which is different from organic dye used in commercial ELISA.

Another aspect of the present invention provides a diagnosis composition for myocardial infarction-related disease, including a complex that includes a quantum dot layer-containing bead particle and an agent for detecting or analyzing a target material, i.e., Sub P or NpY.

A description of the quantum dot layer-containing bead particle included in the diagnosis composition for myocardial infarction-related disease is the same as described above. The term “myocardial infarction-related disease” as used herein refers to necrotic tissues or cells of heart muscles due to acute or chronic cardiovascular stenosis, or refers to all diseases that cause pain due to abnormality in heart muscles by reduction of blood supply to the heart. For example, myocardial infarction-related disease includes myocardial infarction, stable angina, or unstable angina. The term “diagnosis” as used herein refers to identification of the presence of disease and features of its pathophysiology. That is, diagnosis of myocardial infarction-related disease refers to identification of myocardial infarction-related disease such as acute myocardial infarction, stable angina, or angina pectoris. Here, SubP may have an amino acid sequence of SEQ ID NO: 1 and NpY may have an amino acid sequence of SEQ ID NO: 2. The diagnosis composition for myocardial infarction-related disease may include an antibody specific to proteins of SubP or NpY, the proteins themselves, or fragments of the antibody or the proteins, in the complex, so as to enable diagnose of myocardial infarction-related disease. The diagnosis composition for myocardial infarction-related disease of the present invention uses the complex including a quantum dot layer, and accordingly, overcomes a limit of quantification of a commercial kit that is currently available in the market, and has high sensitivity of the quantum dot layer. Thus, in the case of myocardial infarction-related disease, accurate and sensitive detection and measurement of a marker, e.g., SubP, present in a very small amount in the blood are available for better diagnosis of myocardial infarction-related disease. Another aspect of the present invention provides a method of manufacturing an immunoassay kit a complex comprising a quantum dot layer-containing bead particle and an antibody which specifically binds to a target material, and a substrate.

The manufacturing method of the complex will be described as follows. First, the method may include activating a surface of the quantum dot layer-containing bead particle. The term “activation” as used herein refers to the description above. In order to perform such activation, conventional methods, such as use of chemicals, plasma treatment, and ionization treatment, that are known in the art may be used. Following the activation, a reactive group, such as an amino group, a carboxyl group, or a hydroxyl group, may be introduced to the surface of the bead particle at the end. In some embodiments, a surface of a silica bead particle is activated by using a silane coupling agent, thereby introducing an amino group to the surface. Here, the introduced reactive functional group is used to bind the agent and the bead particle.

Next, the method may include binding the surface of the activated bead particle and the agent for detecting or analyzing the target material via a linker-mediated covalent bond. The linker may refer to a general structure capable of connecting at least the bead particle and the agent. The linker may refer to, for example, a structure available for a covalent connection, and may be, for example, an amide bond. The linker may be a cross-linker agent, and examples thereof include polyethylene glycol, aldehyde, isocyanate, maleimide, and glutaraldehyde. In some embodiments, glutaraldehyde may be used as a liker so as to connect the agent and the bead via an amide bond.

In addition, the manufacturing method of the substrate may include activating a surface of the substrate using Parylene A and glutaraldehyde for dense coating of antigens. Following the activation, a reactive group, such as an amino group, a carboxyl group, or a hydroxyl group, may be introduced to the surface of the microplate. In some embodiments, a surface of a polystyrene microplate is activated by using a glutaraldehyde, thereby introducing an amino group to the surface. Here, the introduced reactive functional group is used to bind the antigen (SubP) or antibody (anti-SubP).

Another aspect of the present invention includes a method of diagnosing myocardial infarction-related disease by using an immunoassay kit a complex comprising a quantum dot layer-containing bead particle and an antibody which specifically binds to a target material, and a substrate.

The method of diagnosing myocardial infarction-related disease will be described as follows. First, the method may include measuring expression of SubP and/or NpY at a protein level in a patient's sample by using a complex including a quantum dot layer-containing bead particle and an agent for detecting or analyzing a target material, wherein the agent includes a material specifically binding to the target material and the target material is SubP or NpY. A description of the complex in which the target material is SubP or NpY is as described above. The term “patient” as used herein refers to a subject with suspected acute myocardial infarction may be any subject in which expression of SubP or NpY is changed upon acute myocardial infarction. The term “sample” as used herein refers to a biological material derived from the subject, and examples of the sample derived from the subject include blood, bone marrow, lymph, saliva, tears, urine, mucosal fluid, amniotic fluid, or a combination thereof.

The term “measurement of the protein expression level” as used herein refers to a process of determining the presence of protein expression of Sub P or NpY in a sample and the extent of the protein expression, thereby diagnosing acute myocardial infarction. In some embodiments, the measurement of the protein expression level may be performed by, for example, Western blotting, enzyme linked immunosorbent assay (ELISA), radioimmunoassay (RIA), radioimmunodiffusion, Ouchterlony immunodiffusion, rocket immunoelectrophoresis, histoimmunostaining, immunoprecipitation assay, complement fixation assay, and FACS, or a method using a protein chip, or a combination thereof. However, in terms of the method or an apparatus using the method, the present complex is used to show fluorescent features thereof, instead of a material that is conventionally used in the art. For example, in the case of ELISA, a fluorescent signal obtained by using the present complex is used instead of a fluorescent signal obtained by using an enzyme to measure the protein expression level.

The measurement of the protein expression level may be performed by, for example, ELISA. Various types of ELISA include direct ELISA in which a labeled antibody immobilized onto a solid support is used to recognize an antigen, indirect ELISA in which a labeled antibody is used to recognize a captured antibody immobilized on a solid support which is complex with an antigen, direct sandwich ELISA in which an antibody is used to recognize an antigen captured by another antibody immobilized onto a solid support, and indirect sandwich ELISA in which a secondary antibody is used to recognize an antibody which captures an antigen complexed with a different antibody immobilized onto a solid support (see FIG. 1). That is, as described above, the measurement of the protein expression level may be performed by using conventional ELISA methods, but may be also performed by analyzing the fluorescent signals derived from the present complex, instead of analyzing the fluorescent signals derived from the enzyme.

For example, in the case of sandwich ELISA, sandwich ELISA includes: coating a surface of a solid substrate with an antibody as a primary antibody that specifically binds to protein of SubP or NpY, or a fragment of SubP or NpY; inducing an antigen-antibody reaction upon a contact of the antibody to a blood sample of a normal subject and a subject with suspected acute myocardial infarction; reacting the resultant of the inducing the antigen-antibody reaction with a secondary antibody associated with an enzyme; and detecting activity of the enzyme. Here, examples of the solid substrate include hydrocarbon polymers (e.g., polystyrene and polypropylene), glasses, metals, or gels. For example, the solid substrate may be a microtitre plate.

When the measurement of the protein expression level is performed by direct ELISA, direct ELISA includes: coating a surface of a solid substrate with a primary antibody, an antibody that specifically binds to protein of SubP or NpY, or a fragment of SubP or NpY; inducing an antigen-antibody reaction upon a competitive contact of the antibody to a blood sample of a normal subject and a subject with suspected acute myocardial infarction, or a standard material that is labeled in a constant amount (i.e., a standard peptide associated with an enzyme, a fluorescent organic material, or a nano-fluorescent organic material); and detecting concentration of the resultant of the inducing antigen-antibody reaction directly measured using a microplate reader or a fluorescence reader (see FIG. 1C).

In addition, the measurement of the protein expression level may be, for example, performed by analysis of Western blotting using at least one antibody with respect to the protein. Here, the entire proteins are separated from a sample, are subjected to electrophoresis to separate them by size, and are migrated to a nitrocellulose membrane to thereby react with an antibody and prepare an antigen-antibody complex. The amount of the proteins is identified according to a method of identifying an amount of the antigen-antibody complex by using a labeled antibody, thereby identifying the presence of myocardial infarction-related disease.

Next, the method of diagnosing myocardial infarction-related disease may include comparing the measured protein expression level in the patient's sample with that of a normal control group. The comparing may be performed by examining an amount of the protein expression in a normal control group and an amount of the protein expression in the patient's sample. Here, the protein level may be differently obtained in absolute value of the marker (e.g., μg/Ml) or in relative value (e.g., relative extent of signals).

In addition, the method of diagnosing myocardial infarction-related disease may include determining myocardial infarction-related diseases in the case of higher protein expression level of SubP or NpY in the patient's sample than in the normal control group.

Regarding the determining, in some embodiments the patient may be determined to have acute myocardial infarction when the expression level of SubP is greater than a first predetermined value. Alternatively, a patient is determined to have acute myocardial infarction when the expression level of SubP is greater than a first predetermined value and the expression level of NpY is greater than a second predetermined value. Alternatively, when the expression level of SubP is greater than the first predetermined value or the expression level of NpY is greater than a third predetermined value, the patient may be determined to have acute myocardial infarction, stable angina, or angina pectoris. Alternatively, when the expression level of SubP is less than the first predetermined value and the expression level of NpY is less than the third predetermined value, the patient may be determined to have no acute myocardial infarction, unstable angina, stable angina, nor angina pectoris. Here, the first predetermined value may be about 122 pg/ml, the second predetermined value is about 59 pg/ml, and the third predetermined value is about 40 pg/ml. In some embodiments, blood concentrations of SubP and NpY in a sample of a normal subject and a patient with acute myocardial infarction are compared and analyzed by using the present complex, and as a result, the blood concentration of the patient's sample is significantly higher than that of the normal subject (P<0.0001).

Hereinafter, the present invention is described in greater detail with reference to embodiments. However, the embodiments are for illustrative purposes only and do not limit the scope of the present invention.

### EXAMPLE 1

### Manufacture of a Complex Including a Quantum Dot Layer-Containing Bead Particle, and an Antibody or a Fragment of SubP or NpY

### 1.1 Manufacture and Activation of a Quantum Dot Layer-Containing Silica Bead Particle and Substrate

A silica bead particle containing a quantum dot layer was prepared according to a method described below (hereinafter, the silica bead particle containing the quantum dot layer is referred to as quantum dot-layered silica sphere (SQS) in Examples and drawings in the present specification).

First, 5 Ml of CdSe/CdS-ODA quantum dot solution (2×10⁻⁵M) in a core/shell structure having a surface protected with octadecylamine (ODA) was placed under vacuum to remove a hexane solvent, thereby dispersing in 10 Ml of chloroform.

Afterwards, an excessive amount of methanol solution in which 0.05 M of mercaptopropionic acid (MPA) and 0.06 M of sodium hydroxide were dissolved was added to the solution to be stirred for 30 minutes. When 2 to 3 mL of distilled water was added to the resultant solution, quantum dots were transferred into a water layer. The water layer was separated, and methanol and ethyl acetate were added to the separated water layer so as to recover the quantum dots by centrifugation. These quantum dots were then dispersed in water and a diluted sodium hydroxide solution was used to adjust pH of the solution approximately to 10, thereby manufacturing 100 mL (1×10⁻⁶M) of polyanionic monodispersed quantum dot CdSe/CdS(—SCH₂CH₂CO₂⁻)ₑₓ aqueous solution whose carboxylic acid on the surface of the quantum dot was in —CO2⁻ state. Next, 5 mL of a silica bead solution (DLS size 1.0±0.05 μm, 10 wt %) purchased from Polyscience Company was placed and subjected to centrifugation. Then, the solution was dispersed in 20 mL of methanol. Here, 0.025 mL of aminopropyltrimethoxysilane was added thereto, and refluxed for 10 hours. The solution was cooled and washed about 3 or 4 times with methanol by centrifugation. Lastly, the resulting solution was dispersed in 10 mL of ethanol and then a few drops of dilute hydrochloric acid were added thereto so that the pH of the solution was adjusted to about 4, thereby manufacturing polyanion monodisperse silica bead solution in which amine on the surface of the SQS is in —NH3+ state. The prepared polyanionic silica bead solution was slowly added to the prepared polyanionic quantum dot solution, and the mixture was shaked to mix them each other uniformly. The shaking was stopped at which precipitate was formed, and the solution was vortexed for 1 minute, and then, subjected to centrifugation. Fluorescence was rarely detected in residual solution, and thus, the solution was discarded and the precipitate was dispersed in 400 mL of ethanol, thereby manufacturing a silica bead solution in which the surface is doped with the quantum dot layer. Then, 12 ml of distilled water and 4 ml of thick ammonium solution was added to the manufactured silica bead solution, and stirred. Next, 2 mL of tetraethoxysilane (TEOS) was added to the solution and stirred for 3 hours. Such adding and stirring were repeated two more times, so as to grow a total 6 mL TEOS as a silica layer on the silica bead having the quantum dot layer doped thereon, thereby manufacturing a silica bead in which the quantum dot layer is doped to the inside adjacent to the surface thereof. The solution was separated by centrifugation and the precipitate was washed with ethanol. Afterwards, the solution was separated again by centrifugation and then dispersed in 20 mL of ethanol, thereby manufacturing SQS.

In order to activate SQS, 2 mL of the SQS solution was diluted to 20 mL. 0.4 mL of concentrated aqueous ammonia was added thereto and then stirred. Next, 0.93 ml of amino propyl trimethoxy silane were added to the mixture and then stirred at a temperature of 20° C. for 14 hours. The mixture was washed twice with ethanol using centrifugation, and finally, was dispersed in 10 mL of ethanol. Here, the concentration of the quantum dot contained therein reached 1 mM.

### 1.2. Binding of Antibody with SQS Via Linker

Glutaraldehyde was used as a cross-linker which connects SQS with activated NH₂ and an amine group of an antibody, so as to connect with a target peptide (i.e., a fragment of SubP or NpY) (100 nM, monoclonal, biorbyt) or an antibody (50 nM, monoclonal, PHOENIX PHARMACEUTICALS, INC.). 100 nm and 300 nm of SQS-NH₂ (at a concentration of 25 nM of contained quantum dots) separated from ethanol were each dissolved in a carbonate buffer (50 mM, pH 9.6) added glutaraldehyde (2.5%), and reacted at a temperature of 37° C. for 2 hours. Then, glutaraldehyde that was not bound to SQS-NH₂ was removed by centrifugation (at 10,000 g for 15 minutes at a temperature of 4° C.).

Table 2 below shows the results of sizes of complex, in which 100 nm of the SQS-NH₂ and antibodies were bound, measured by using Zeta PAL (Brookhaven Instrument Co., USA). The size of the antibody was about 15 nm to 20 nm, and in this regard, the coupling ratios of 1:2 and 1:4 are both referred that two or more antibodies were bound to 1 SQS.

TABLE 2

| | Coupling ratio of SQS to Ab | | |
|---|---|---|---|
| | SQS only | 1:2 | 1:4 |
| Mean diameter (nm) | 168 ± 27 | 211 ± 27 | 243 ± 19 |
| Relative variance | 1.1 | 1.3 | 0.66 |

Table 3 below shows the results of measuring the size of the composite to which SQS-NH2 of 200 nm in size and antibodies are bound. Here, the coupling ratios of 1:2 and 1:4 are both referred that two or more antibodies were bound to 1 SQS.

TABLE 3

| | Coupling ratio of SQS to Ab | | |
|---|---|---|---|
| | SQS only | 1:1 | 1:2 |
| Mean diameter (nm) | 207 ± 25 | 220 ± 27 | 236 ± 19 |
| Relative variance | 1.1 | 1.3 | 0.66 |

### EXAMPLE 2

### Optimazation of Immunoassay kit

### 2.1. Optimazation of Substrate

To determine the effect of Parylene A coating on the density of SubP or anti-SubP antibody, we performed sandwich ELISA and competitive SQSLISA for SubP using both polystyrene microplates and Parylene A-modified plates. Sandwich SQSLISA was used to analyze the sensitivity of Parylene A-coated plates. Parylene A-coated plates exhibited approximately 2-fold higher PL intensity than the polystyrene plates in the range of 0.01-100 ng/mL of SubP (p<0.05, See FIG. 3). When used for competitive SQSLISA, the Parylene A-coated plates produced PL intensities that were about 5-fold higher at 0.01 ng/mL and 1.5-fold higher at 10 ng/mL SubP (data not shown). The difference of density for the coated antibody possibly gave different results.

### 2.2. Optimazation of the SQS

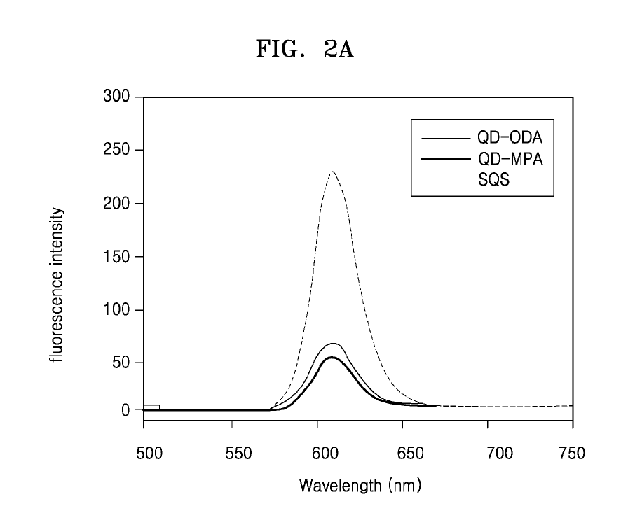

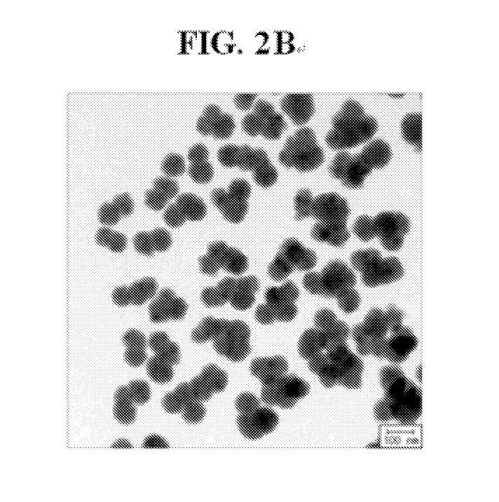

To prepare a highly sensitive signaling SQS, we modified our previous synthetic method. Roughly, 80, 300, and 800 nm-sized SQS particles showed 2.4-, 3.2-, and 2.1-fold enhanced PL, respectively, relative to a QD-MPA solution at a constant QD concentration. Regarding the result, even though the ˜80 nm-sized SQS should display the lowest light scattering effects and consequently the highest PL enhancement among the three samples, 300 nm-sized SQS particles showed the highest PL enhancement. It was assumed that the ˜80 nm-sized SQS cannot avoid aggregation between themselves during synthesis because of the high surface energy resulting from their small size and aggregation increases the light scattering effect. On the other hand, it is relatively easy to control the aggregation of the ˜300 and ˜800 nm-sized SQS. Therefore, we expected that the reduction of aggregation in ˜80 nm-SQS can lead to the highest PL enhancement among the three sizes of SQS. To reduce aggregation, the silica encapsulation reaction of SQ assembly was performed in a 5-fold diluted solution ([QD]=5×10⁻⁸ M), relative to a previously reported protocol. Periodic sonication of the reaction mixture further reduced aggregation. Sonication aided in the dispersion of relatively large (submicrosized) particles whereas it promoted the aggregation of relatively small nanoparticles (<˜20 nm). Therefore, we sonicated the solution after 1 h, when the thin silica shield had already formed on the SQ assembly. Resultantly, the synthesized SQS (about 100 nm diameter) exhibited 5.1- and 4.2-fold higher PL intensities than QD-MPA and the purchased QDODA, respectively, at a constant QD concentration (FIG. 2A).

### 2.3. Optimization of the Immunoassay Condition

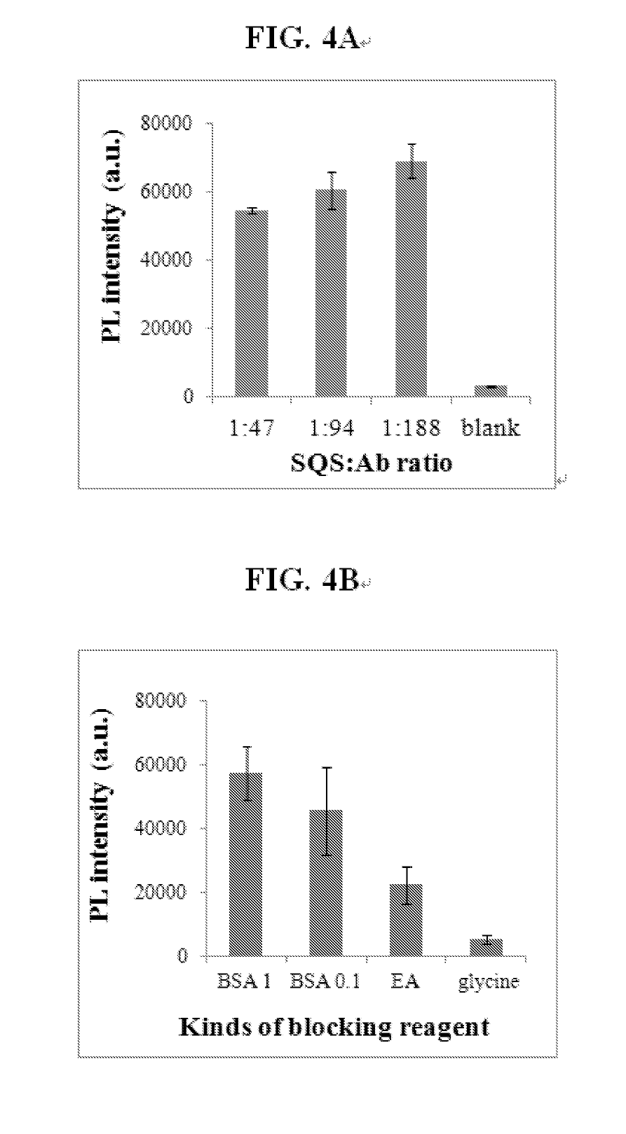

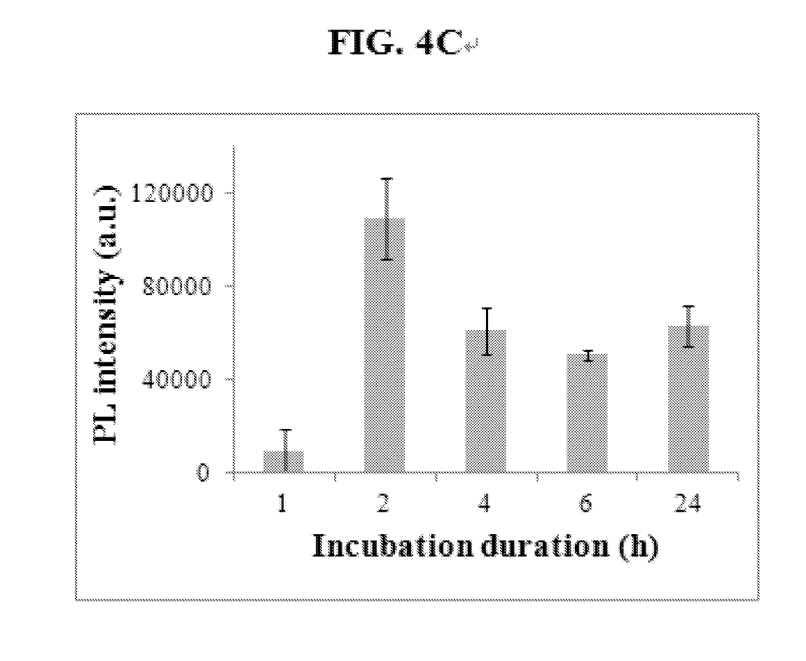

The assay conditions and protocols were optimized to maximize assay sensitivity. To optimize the binding ratio between SQS and the SubP monoclonal antibody, different concentrations of antibody (75, 150, and 300 nM in 200 μL) and the activated SQS-NH2 (1.6 nM, 200 μL) were mixed and incubated with slow shaking (1400 rpm) on a thermo mixer (Eppendorf AG, Hamburg, Germany) at 37° C. for 2 h to form SQS and antibody conjugation. After optimization of the reaction ratio, 300 nM of monoclonal anti-SubP antibody was used for the preparation of the SQS-labeled Ab. The SQS-labeled Ab was separated by centrifugation at 10,000 g for 10 min (4° C.) and dispersed in PBS. As a result, the optimal ratio of SQS and anti-SubP antibody for conjugation was determined. The molar ratio of 1:188 for SQS/Ab showed the highest PL intensity among the three tested ratios in the assay (See FIG. 4A). Increasing the molar ratio more than 1:188 did not yield any further increase in PL intensity.

In addition, various types and concentrations of blocking reagents were tested, and 1 mg/mL of BSA produced the results with the highest sensitivity for SQSLISA (FIG. 4B).

In addition, Various incubation time for binding Sub P on the plate to optimize immunoassay condition was tested, and the incubation of SubP on the plate for 2 h resulted in the highest intensity (FIG. 4C).

### EXAMPLE 3

### Immunological Assay on Blood Samples Using Composite

### 3.1. Preparation of Sample and Assay Method Using the Sample

In order to measure concentration of SubP or NpY in a blood sample, a commercial kit (PHOENIX PHARMACEUTICALS, INC.) and the SQS-Ab complex manufactured in Example 1 were used to carry out experiments according to sandwich ELISA.

The experiments according to sandwich ELISA using the commercial kit were carried out for samples in a total of three groups. In detail, blood samples were prepared by an experimental subject with myocardial infarction and unstable angina (40 people), another experimental subject with stable angina (40 people), and a normal control subject (80 people).

The experiments according to sandwich ELISA using the commercial kit were carried out for samples in a total of four groups. In detail, blood samples obtained from normal people (29 people), patients with acute myocardial infarction (30 people), patients with unstable angina (28 people), and patients with angina pectoris (30 people) were tested.

500 μl of blood samples of normal people and patients with cardiovascular disease, which were obtained from School of Medicine, University of Korea, were diluted two-fold with 10% formic acid buffer to extract peptides by using Oasis HLB 1 cc (30 mg) Extraction Cartridges Solid phase Extraction Kit (Waters, Ireland). Meanwhile, endogenous peptides in serum and plasma were separated from proteins by using 30 kDa Molecular cut-off filter (Millipore, USA). A solution containing these extracted peptides was dried using a N₂ Evaporator or a Freeze-Dryer. Afterwards, in consideration of immunological analysis, the solution was dissolved in 50 μl of assay buffer (provided by each ELISA kit manufacturing company) for analysis, or 25 μl of the serum or plasma was diluted two-fold in PBS for direct analysis.

### 3.2. Measurement of Concentration of NpY by Using Commercial Kit

In the preparation of standard solutions for a calibration curve, 1,000 ng/ml of a stock solution was diluted with a 1× assay buffer solution and adjusted to prepare standard solutions at concentrations of 100, 10, 1, 0.1, and 0.01 ng/ml. Then, 50 μl of a positive control group was prepared as a control group. Into each well of the microplate coated with secondary antibodies, the control group and the standard solution were aliquoted in duplicate and two-fold diluted samples were aliquoted triplicate. Afterwards, 25 μl of primary antibodies (rabbit anti-peptide IgG) (PHOENIX PHARMACEUTICALS, INC.) obtained by which artificial NpY was injected in a rabbit and 25 μl of biotinized peptide were aliquoted to the microplate, and then, incubated at a speed of 300 to 400 rpm at room temperature for 2 hours. Then, the microplate was washed four times with a washing buffer and 100 μl of streptavidin-horseradish peroxidase were added thereto. Then, the microplate was shaken (at a speed of 300 to 400 rpm) for 1 hour. Afterwards, the microplate was washed in the same manner four times with a washing buffer, and 100 μl of TMB substrate solution was atomized thereto. The microplate was placed at room temperature for 1 hour for reaction. Lastly, 100 μl of a stop solution (2N HCl) was used to terminate the reaction. The microplate was exposed to a wavelength of 450 nm to measure OD values using a microplate reader.

### 3.3. Measurement of Concentration of SubP by Using Commercial Kit

In order to perform an experiment according to ELISA, a commercial kit from R&D system was used, and the competitive binding principle was on the basis of the experiment. In the preparation of standard solutions for a calibration curve, 50 ng/ml of a SubP standard solution were diluted with 1 ml of Calibrator Diluent RD5-45. Then, 300 μl of the diluted solution was subjected to serial dilution with 1 ml of Calibrator Diluent RD5-45 to prepare the standard solutions at concentrations of 2,500, 1,250, 625, 312, 156, 78, 39, and 0 pg/ml. 213 to 540 pg/ml, 517 to 982 pg/ml, 1224 to 2217 pg/ml of a control solution and zero standard well were set as a control group, and the standard solutions, the control group, and the zero standard well were used in duplicated) in the experiment. A serum sample was diluted two-fold and used in triplicate in the experiment. To an ELISA microplate coated with goat anti-mouse polyclonal antibodies and included in the kit, 50 μl of the standard solutions, the control solution, and the diluted serum sample were each aliquoted. Then, to each well of the microplate. 50 μl a primary antibody solution (i.e., mouse monoclonal antibody with respect to SubP) and 50 μl of a SubP-conjugated HRP were sequentially aliquoted, and the microplate was covered by an adhesive strip. The microplate was incubated in a microplate shaker at a speed of about 500±50 rpm for 3 hours. Afterwards, each well of the microplate was washed four times with a washing buffer. 200 μl of TMB substrate solution was aliquoted thereto, and incubated for 30 minutes. Lastly, 50 μl of a stop solution (2N sulfuric acid) was aliquoted thereto. Here, it was confirmed that the blue color of the solution was changed to yellow. Then, the microplate was exposed to a wavelength of 450 nm to measure OD values using a microplate reader.

### 3.4. Measurement of Concentration of SubP by Using SQS-Ab Complex (SQSLISA: Silica Based Nano Quantum dot Sphere Linked Immuno-AdSorbent Assay)

On a 96-well microplate coated with parylene A, parylene A was activated by ethanol in which 10% glutaraldehyde was dissolved. Then, Sub P was incubated for 2 hours so as to coat the 96-well microplate, thereby manufacturing the microplate blocked by bovine serum albumin (BSA). Then, PBS in which molecules of SQS-SubP antibodies were dissolved was incubated with a patient's serum sample that was diluted in PBS to precede a binding reaction between SubP or standard SubP present in the serum and the SQS-SubP antibodies. The SQS-SubP antibodies were separated from the complex of the SQS-SubP antibodies and SubP by centrifugation, and then, placed in the pre-built microplate wells. The microplate was incubated for 3 hours. When the concentration of SubP was high in the serum, the SQS-SubP antibodies were already saturated in terms of binding to SubP. Accordingly, the antibodies SQS-SubP antibodies did not bind to SubP in the microplate anymore. That is, the higher the blood concentration, the lower the fluorescence intensity of ELISA was extracted.

Afterwards, the microplate was lightly washed with PBS in which 0.1% Tween was added. By using a multi-label plate reader, fluorescence at an excitation wavelength of 486 nm and an emission wavelength of 620 nm was measured.

It was found that the immunological assay using the complex of the present invention had very high quantitation and reproducibility. When 100 nm SQS was used, as shown in Table 4 below, the immunological assay was resulted with 120% of accuracy and 10% or less of precision, meaning very high quantitation and reproducibility. In addition, the immunological assay of the present invention had a wide dynamic range compared to that of other commercial immunological assay methods (˜10⁵).

TABLE 4

| | Intraday assay | | Interday assay | |
|---|---|---|---|---|
| | Accuracy | Reproduc- | Accuracy | Reproduc- |
| Parameter | (%) | ibility (%) | (%) | ibility (%) |
| QC conc. | 120 | 9.9 | 100 | 10.1 |
| (0.1 ng/ml) | | | | |

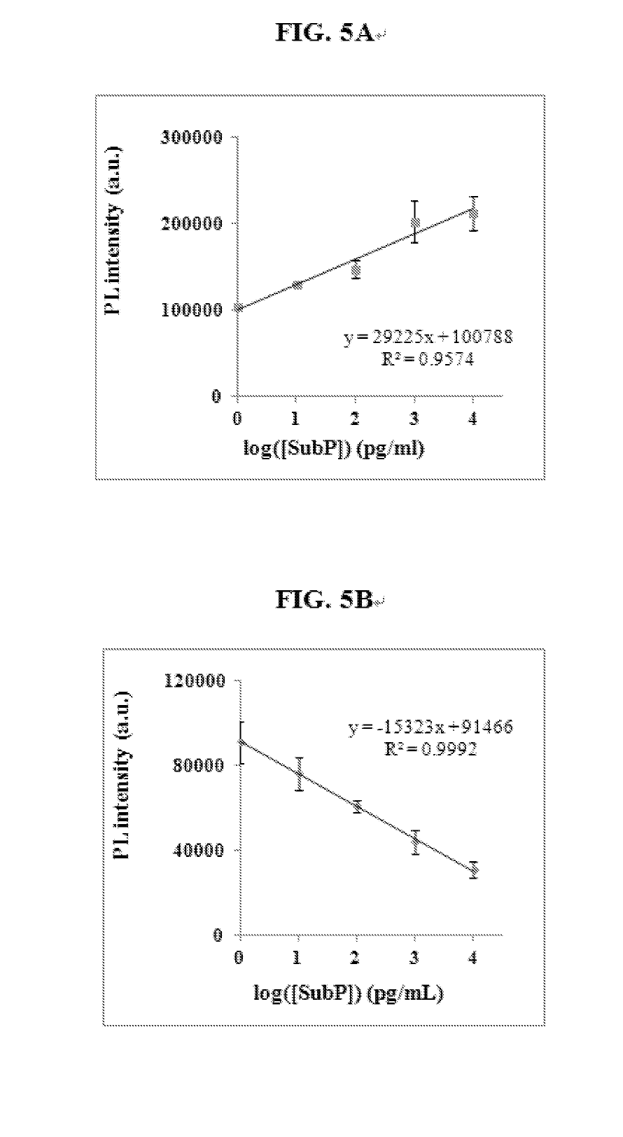

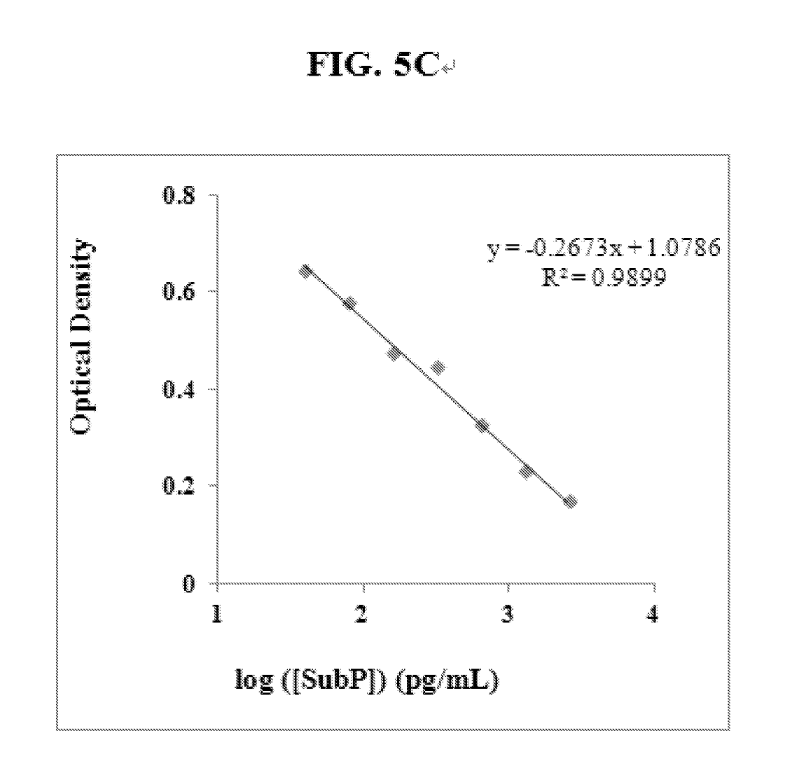

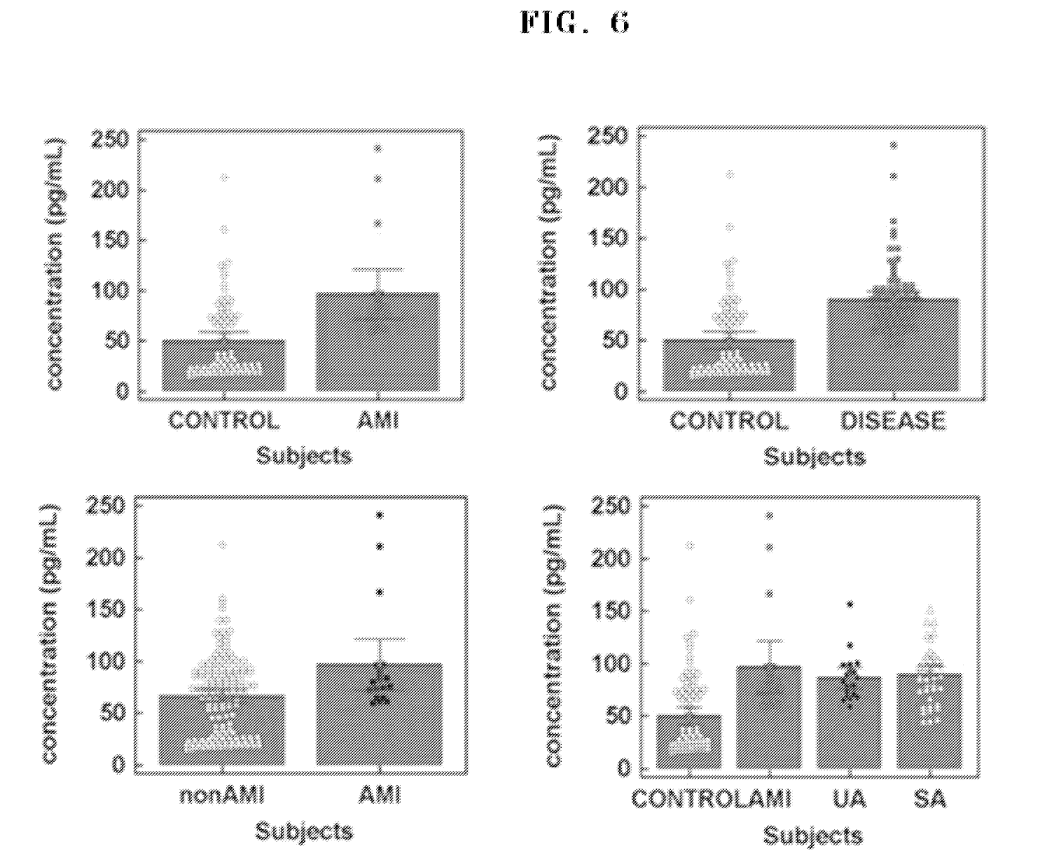

We compared calibration curves between SQSLISA (FIGS. 5A,B) and ELISA tested (FIG. 5C). For SQSLISA, calibration curves were generated for both direct (FIG. 5A) and competitive SQSLISA (FIG. 5B). The SQSLISAs gave wider dynamic range (10⁴ orders) than ELISA (10³ orders). The correlation factor for ELISA was 0.9899 which was higher than the direct SQSLISA and lower than competitive SQSLISA. The competitive SQSLISA showed the highest linearity (0.9992) and the best reproducibility (CV<7.4%) among three calibration curves.

### EXAMPLE 4

### Diagnosis of a Patient with Suspected Myocardial Infarction-Related Disease by Using SubP and NpY

### 4.1. Analysis of NpY Protein Concentration in a Sample by Using a Commercial Kit

First, a commercial kit was used to analyze concentrations of SubP and NpY in blood samples from a normal subject and a patient with cardiovascular disease, according to the ELISA method described above.

FIG. 6 shows the results of comparative concentrations of NpY in blood samples from a normal subject and a patient with cardiovascular disease. FIG. 6(a) shows NpY concentrations in the case of comparing a normal subject with a patient with acute myocardial infarction (AMI), wherein *P value<0.0001, Cut-off value>40 pg/ml. FIG. 6(b) shows NpY concentrations in the case of comparing a normal subject with a patient with cardiovascular disease (e.g., unstable angina (UA), stable angina (SA), and acute myocardial infarction (AM I)), wherein *P value<0.0001, Cut-off value>59 pg/ml. FIG. 6(c) shows NpY concentrations in the case of comparing a patient with AMI and a patient with disease other than AMI (non-AMI). FIG. 6(d) shows NpY concentrations in the case of comparing a normal subject with a patient with each disease (i.e., AMI, UA, and SA).

On the basis of the results, Table 5 below shows significance values (p, %), which were statistically calculated based on t-test and area under curve (AUC) values obtained by calculating values in the receiver operating characteristic (ROC) curve, and positive prediction rates (PPR, %) and negative prediction rates (NPR, %).

TABLE 5

| | Sensitivity | Specificity | cut-off | | PPR | NPR |
|---|---|---|---|---|---|---|
| | (%) | (%) | (pg/mL) | p value | (%) | (%) |
| control vs. | 100 | 59 | >40 | <0.0001 | 70 | 100 |
| disease | | | | | | |
| control vs. | 100 | 64 | >59 | <0.0001 | 71 | 88 |
| AMI | | | | | | |
| AMI vs. | 100 | 41 | >59 | 0.0036 | 20 | 100 |
| non AMI | | | | | | |

### 4.2. Analysis of SubP Concentration by Using a Commercial Kit

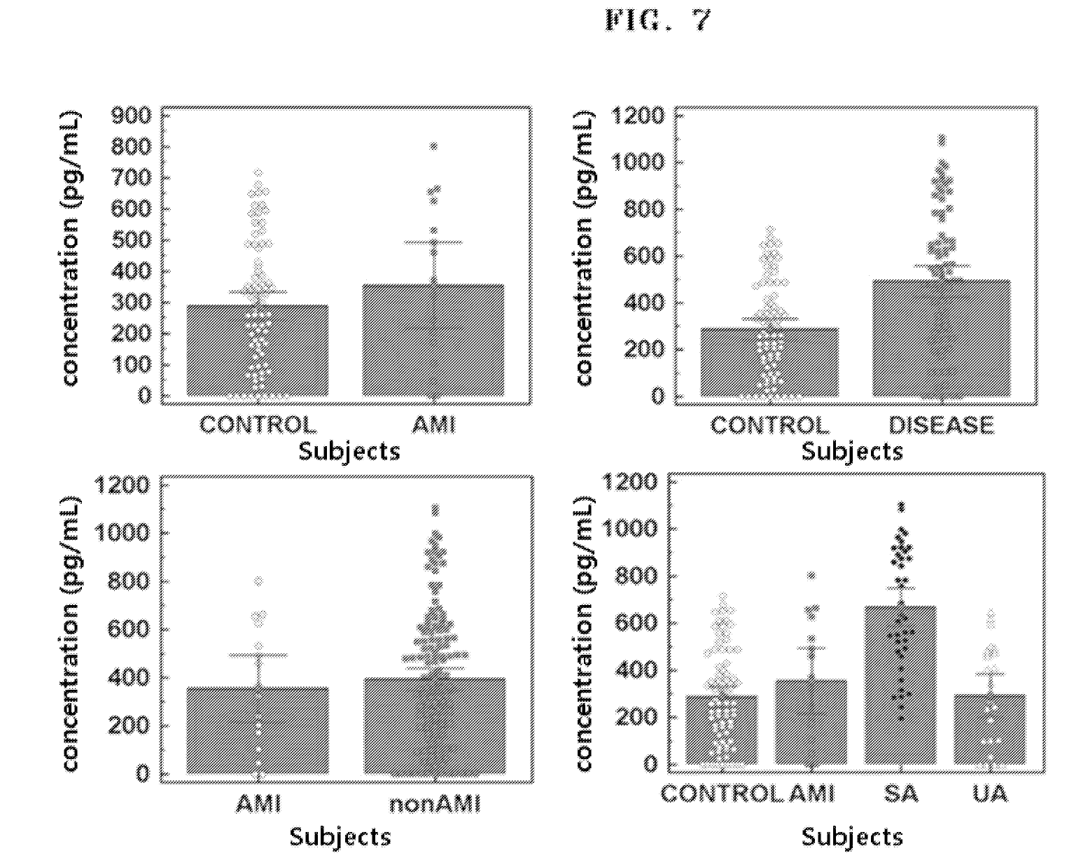

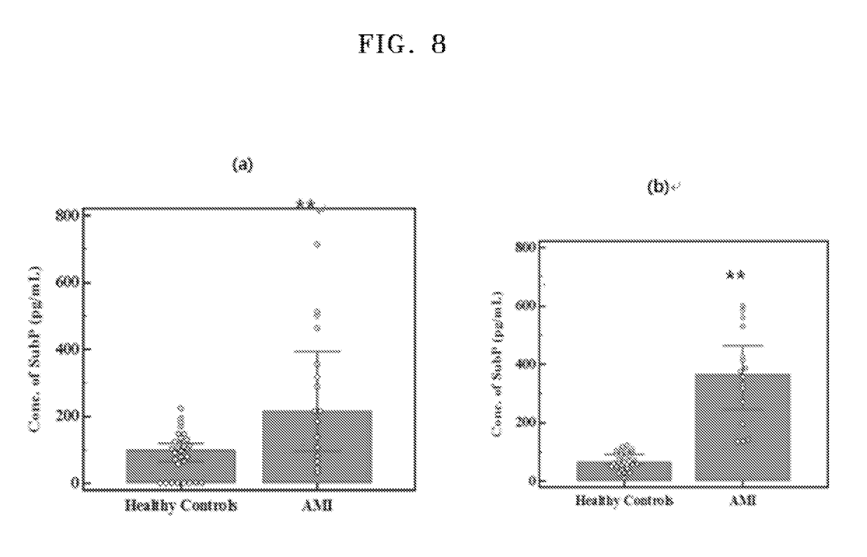

The results of analyzing concentration of SubP by using a commercial kit were shown in FIG. 7. FIG. 7(a) shows SubP concentrations in the case of comparing a normal subject with a patient with AMI, wherein Cut-off value>364 pg/ml which was not statistically significant. FIG. 7(b) shows SubP concentrations in the case of comparing a normal subject with a patient with cardiovascular disease ((e.g., unstable angina (UA), stable angina (SA), and AMI), wherein *P value<0.0001, Cut-off value>400 pg/ml. FIG. 7(c) shows SubP concentrations in the case of comparing a patient with AMI and a patient with disease other than AMI (non-AMI). Here, it was noticeable that NpY concentration in a sample from a patient with UA was significantly higher than that in a sample from a normal subject and a patient with disease other than UA (p<0.0001). On the basis of the results, Table 6 below shows significance values which were statistically calculated based on t-test and AUC values obtained by calculating values in the ROC curve, and PPR and NPR.

TABLE 6

| | Sensitivity | Specificity | cut-off | | PPR | NPR |
|---|---|---|---|---|---|---|
| | (%) | (%) | (pg/mL) | p value | (%) | (%) |
| control vs. disease | 40 | 94 | 547 | <0.0001 | 76 | 69 |
| control vs. AMI | 40 | 97 | 524 | <0.0001 | 29 | 88 |

### 4.3. Analysis of the Concentration of SubP by Using the Complex

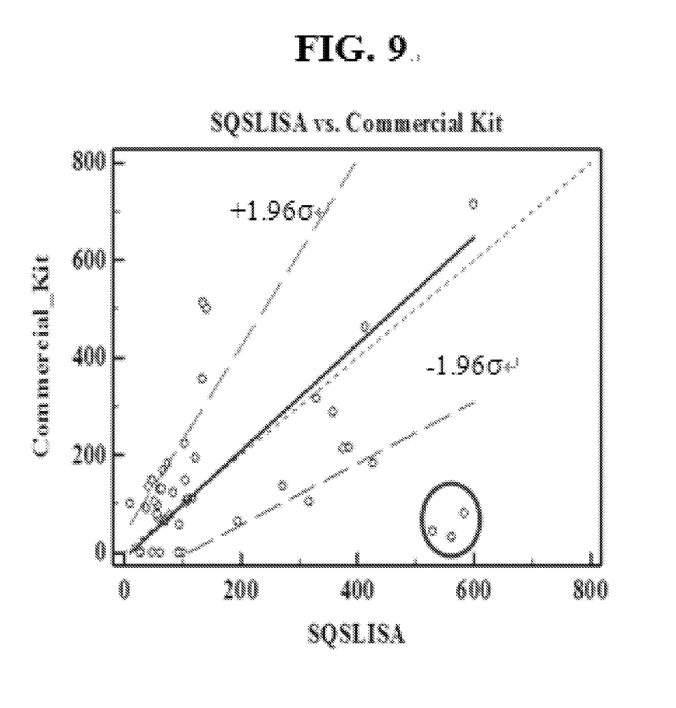

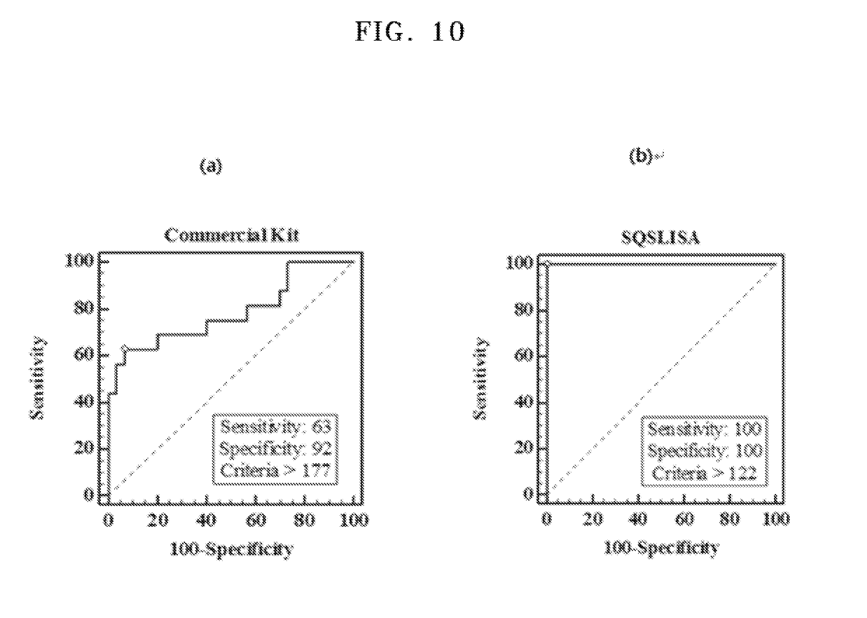

Analysis of blood concentration of SubP using SQS was shown in FIGS. 9 10, and in the case of a patient with acute myocardial infarction, the blood concentration of the patient was significantly higher than that of a normal person (i.e., a control group) (p<0.0001). AUC obtained by calculated ROC curve, its significance statistically based on t-test were shown in FIG. 9.The table 7 showed the comparison of commercial ELISA kit and SQSLISA.

TABLE 7

| | Commercial ELISA kit | SQSLISA |
|---|---|---|
| Dynamic range (pg/mL) | 39-2500 | 1-10000 |
| LOQ (pg/mL) | 30 | 1 |
| Linearity (R²) | 0.9899 | 0.9992 |
| Reproducibility | <8.4%/<15% | <7.4%/<11% |
| (intra-day/inter-day) | | |
| Recovery | 85-117% | 92-100% |
| Cut-off value for | 177 | 122 |
| AMI diagnosis (pg/mL) | | |
| Sensitivity | 63% | 100% |
| Specificity | 92% | 100% |

As a result, SubP measured by SQSLISA showed the sensitivity of 100%, and the specificity of 100% (see Table 7). That is, two markers of SubP and NpY were found to be very sensitive so that they may be used in a very useful way as markers for diagnostic acute myocardial infarction.

As a result, SubP and NpY measured by commercial ELISA showed the ROC of at least 0.8, the sensitivity of 100%, and the specificity of at least 64% (see Table 8 below). That is, two markers of SubP and NpY were found to be very sensitive so that they may be used in a very useful way as markers for diagnostic acute myocardial infarction.

TABLE 8

| | | SubP- | NpY |
|---|---|---|---|
| | SubP- | commercial | commercial |
| Parameter | SQSLISA | ELISA | ELISA |
| Cut-off value | 122 pg/ml | 177 pg/ml | 59 pg/ml |
| AUC | 1.00 ± 0.000^{a,b} | 0.681 ± 0.045^{a,b} | 0.799 ± 0.047^{a,b} |
| Specificity | 100% | 92% | 64% |
| Sensitivity | 100% | 63% | 100% |

AUC, area under the curve
^{a}AUC ± standard error
AUC comparison between SubP and NpY at
^{b}All p < 0.05

The sensitivity and specificity of each assay were calculated using the cut-off values.

As described above, according to the one or more of the above embodiments of the present invention, provided are a complex including a quantum dot layer-containing bead particle and an agent for detecting or analyzing a target material, and a composition including the complex, to thereby provide fast and highly accurate analysis results in terms of detecting or analyzing the target material. In addition, according to the one or more of the above embodiments of the present invention, provided are a diagnosis composition for myocardial infarction-related disease and a method of diagnosing acute myocardial infarction so as to have high specificity and sensitivity with respect to myocardial infarction-related disease as well as acute myocardial infarction from a patient with suspected cardiovascular disease. Thus, the method may be used for early diagnosis and determination of the disease.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A complex comprising:
a quantum dot layer-containing bead particle; and
an agent for detecting or analyzing a target material.

2. The complex of claim 1, wherein the bead particle has a diameter in a range of about 80 nm to about 300 nm.

3. The complex of claim 1, wherein the bead is formed of at least one selected from silica, titanium, zirconia, and zeolite.

4. The complex of claim 1, wherein the agent is a target material or a fragment thereof, an antibody, a peptide, a nucleic acid or a derivative of the antibody, the peptide, or the nucleic acid, which specifically binds to the target material.

5. An immunoassay kit comprising:
a complex comprising a quantum dot layer-containing bead particle and an antibody which specifically binds to a target material; and a substrate.

6. The immunoassay kit of claim 5, wherein the bead particle has a diameter in a range of about 80 nm to about 300 nm.

7. The immunoassay kit of claim 5, wherein the substrate is coated with Parylene A.

8. The immunoassay kit of claim 5, wherein the complex is prepared by reacting a quantum dot layer-containing bead particle and antibody at a moral ratio of about 1:150 to about 1:190.

9. The immunoassay kit of claim 5, which further comprises a blocking agent, wherein the blocking agent is BSA in a range between about 0.5 mg/mL to about 2 mg/mL

10. The immunoassay kit of claim 5, wherein the target material is Sub P, NpY or NT-proBNP, and wherein the immunoassay kit is used for the diagnosis of acute myocardial infarction.

11. A method of diagnosing myocardial infarction-related disease, the method comprising:
measuring an expression level of proteins of Sub P and/or NpY in a patient's sample by using the immunoassay kit of claim 5;
comparing the measured expression level with that of a normal control group; and
determining that the patient has myocardial infarction in the case of higher expression level of the proteins in the patient's sample than in the normal control group.

12. The method of claim 11, wherein a patient is determined to have acute myocardial infarction when the expression level of SubP is greater than a first predetermined value.

13. The method of claim 12, wherein the first predetermined value is about 122 pg/ml.

14. The method of claim 11 wherein measuring of the expression level of SubP proteins is performed by using the immunoassay kit of claim 5, and the measuring of the expression level of NpY proteins is performed by using a laboratory immunoassay kit manufactured by Phoenix Pharmaceuticals Inc.

15. The method of claim 14, wherein a patient is determined to have acute myocardial infarction when the expression level of SubP is greater than a first predetermined value and the expression level of NpY is greater than a second predetermined value.

16. The method of claim 14, wherein a patient is determined to have cardiovascular disease including acute myocardial infarction, stable angina, or unstable angina when the expression level of SubP is equal to or greater than a first predetermined value, or the expression level of NpY is equal to or greater than a third predetermined value.

17. The method of claim 14, wherein a patient is determined to have no cardiovascular disease when the expression level of SubP is smaller than a first predetermined value, and the expression level of NpY is smaller than a third predetermined value.

18. The method of claim 15, wherein the first predetermined value is about 122 pg/ml, and the second predetermined value is about 59 pg/ml.

19. The method of claim 16, wherein the first predetermined value is about 122 pg/ml, and the third predetermined value is about 40 pg/ml.

20. The method of any one of claims 17, wherein the first predetermined value is about 122 pg/ml, and the third predetermined value is about 40 pg/ml.
